(19) United States
(12) Patent Application Publication
(10) Pub. No.: US 2008/0156333 A1
(43) Pub. Date: Jul. 3, 2008

(54) RADIO LUCENT RESTRAINING DEVICE

(76) Inventors: David Wolf Galpern, Brooklyn, NY (US); Manuel A. Guerrero, New York, NY (US)

Correspondence Address: MEREDITH & KEYHANI, PLLC, 330 MADISON AVE., 6TH FLOOR, NEW YORK, NY, 10017, US

(21) Appl. No.: 11/942,193

(22) Filed: Nov. 19, 2007

Related U.S. Application Data
(60) Provisional application No. 60/882,995, filed on Dec. 31, 2006.

(51) Int. Cl. A61F 5/37

(52) U.S. Cl. 128/846

(57) ABSTRACT

An apparatus for stabilizing a portion of a patient, the apparatus comprising: at least one attachment means having a flexible elongated portion and at least one bead attached to the flexible elongated portion; a radio lucent portion, the radio lucent portion having a multitude of raised portions forming a multitude of receiving cavities, each of the receiving cavities shaped to removably receive at least a portion of the attachments means, for example the bead or the flexible elongated portion.

This application claims priority to provisional patent application 60/882,995 filed Dec. 31, 2006 entitled “Radiographically Lucent Hand Restraining Device.”

The present invention relates to apparatus for restraining a portion of a patient during surgery that does not obstruct x-ray machines. Physicians, during surgery, often utilize x-ray machines to see the internal workings of the human body to be operated on and determine the best strategy.

While it is desirable to x-ray a portion of a patient during surgery, it is also desirable to stabilize that same portion of the patient for surgery, such as the patients hand or other extremity. One problem is that restraining devices can interfere with the ability of the surgeon to x-ray without having to remove the restraining device, take an x-ray and reattach the restraining device. This is time consuming and also may result in a different position of the portion of the patient to be operated on requiring the surgeon to reassess and compare the x-ray to the current and changed position of the hand.

Accordingly, the present invention solves these problems by providing a restraining device that is radio lucent. In this way, the portion of the patient, such as the hand, may be restraining and the x-ray machine may also provide guidance to the surgeon at the same time without requiring the surgeon to remove the portion of the patient.

These and other features, aspects and advantages of the present invention will become better understood with reference to the following description and claims.

### SUMMARY OF THE INVENTION

The present invention relates generally to apparatus for restraining a portion of a patient while allowing for x-raying.

According to one embodiment of the present invention, an apparatus for stabilizing a portion of a patient is provided, the apparatus comprising: at least one attachment means having a flexible elongated portion and at least one bead attached to the flexible elongated portion; a radio lucent portion, the radio lucent portion having a multitude of raised portions forming a multitude of receiving cavities, each of the receiving cavities shaped to removably receive at least a portion of the attachments means.

According to another embodiment of the present invention, a radio lucent method of stabilizing a portion of a patient during surgery, the method comprising the steps of: providing a portion of a patient for operating on; placing a radio lucent portion on an operating field, the radio lucent portion having a multitude of raised portions forming a multitude of receiving cavities, the receiving cavities shaped to removably receive one of the beads; placing the portion of a patient on the radio lucent portion; providing at least one attachment means having a flexible elongated portion and at least one beads attached to the flexible elongated portion; removably attaching a portion of the patient to the radio lucent portion by placing at least one attachment means around the portion of a patient and removably attaching at least a portion of the attachment means to one of the multitude of receiving cavities.

According to yet another embodiment of the present invention, a radio lucent kit for stabilizing a portion of a patient during surgery, the kit comprising: at least one attachment means having a flexible elongated portion and at least one bead attached to the flexible elongated portion; a radio lucent portion, the radio lucent portion having a multitude of raised portions forming a multitude of receiving cavities, the receiving cavities shaped to removably receive at least a portion of the attachments means.

These and other features, aspects and advantages of the present invention will become better understood with reference to the following description and claims.

### BRIEF DESCRIPTION OF THE DRAWINGS

FIG. 1 depicts the present invention;

FIG. 2 depicts the present invention; and

FIG. 3 depicts the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

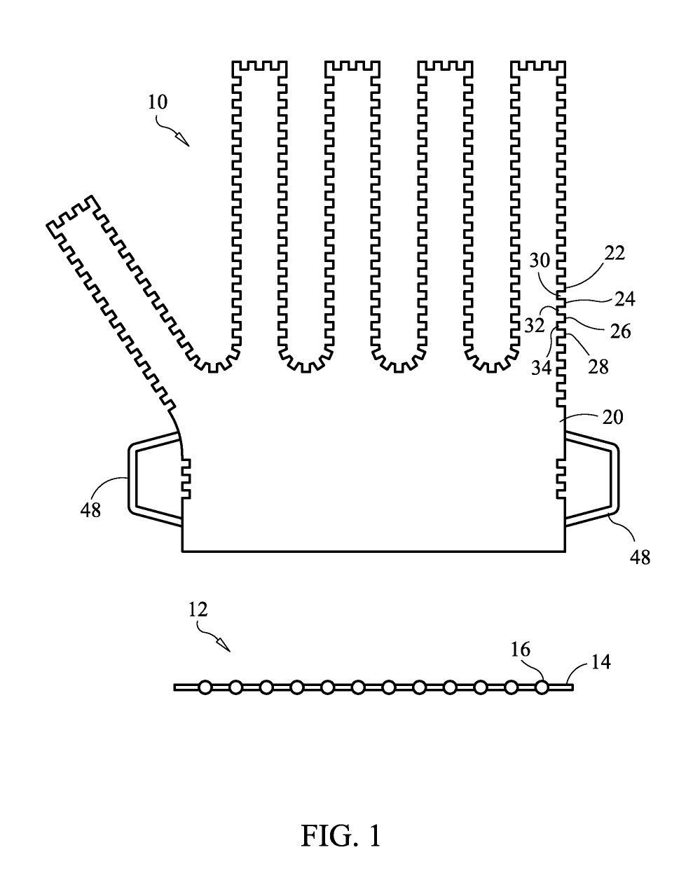

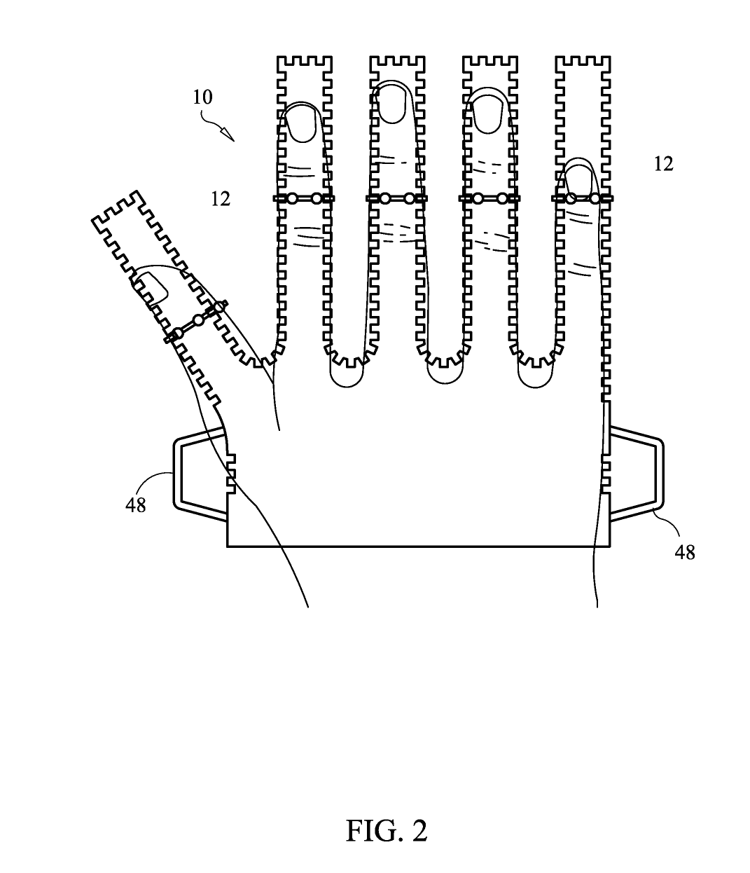

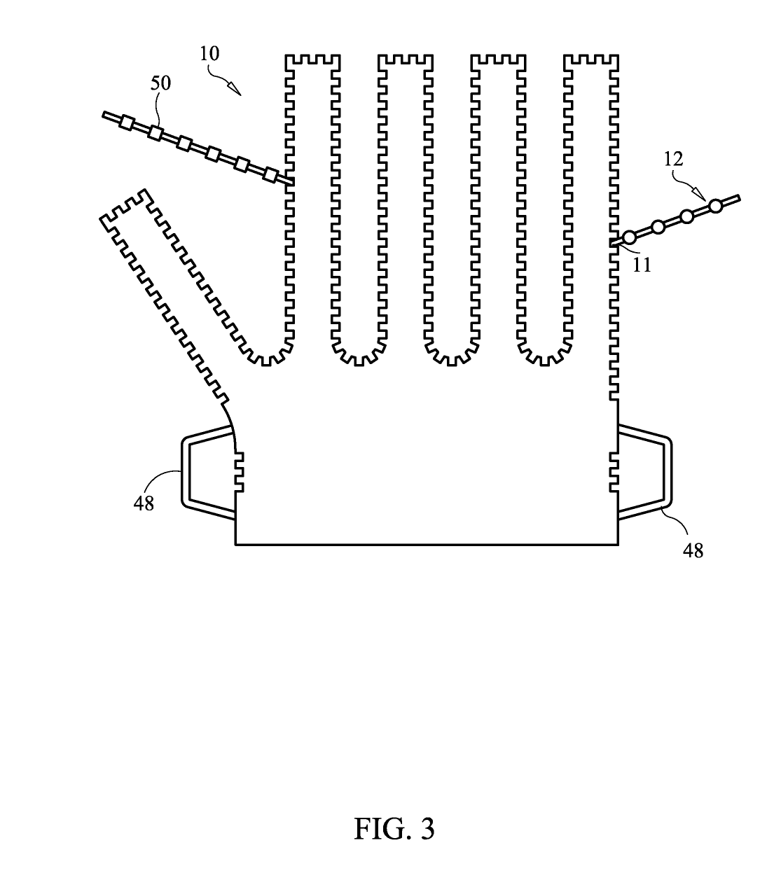

The term radio lucent refers to materials which are transparent to X-rays or other forms of radiation. The term radio lucent is interchangeable with the term radiographically lucent. Examples of radio lucent materials, without limitation, are plastic, carbon reinforced plastic, thermoplastic resin, thermoplastic polymers, Polyetherimides, Polyphenylene sulfides and fiberglass. With reference to FIG. 1, an apparatus (10) for stabilizing a portion of a patient is provided, the apparatus comprising: at least one attachment means (12) having a flexible elongated portion (14) and at least one bead (16) attached to the flexible elongated portion (14); a radio lucent portion (20), the radio lucent portion having a multitude of raised portions (e.g. 22, 24, 26, 28) forming a multitude of receiving cavities (30, 32 and 34), each of the receiving cavities (30, 32 and 34) shaped to removably receive at least a portion of the attachments means. According to the example shown, the flexible elongated portion (14) would fit into the multitude of raised portions (e.g. 22, 24, 26) and the bead (16) would prevent the elongated portion (14) from moving, thus restraining the hand. There may also be at least one slit (48). The slit (48) may be used to tape the apparatus to the operating table. Typically the slit (48) would be along a side of the flexible elongated portion. FIG. 2 depicts a patients hand attached to the apparatus. As shown, the hand (30) sits on the radio lucent portion and the attachment means (12) wraps around each of the fingers to secure the fingers. The receiving cavities (30, 32 and 34) as shown are rectangular. It is envisioned, though, that they may be three quarters circles shaped to receive circular beads. The beads may also be rectangular (as shown in FIG. 3, bead (50)) or any other shape without departing from the claimed invention. In the preferred embodiment depicted, the receiving cavities are shaped to fixedly removably receive the elongated portion between each of the beads to provide stabilization of the hand of the patient. The receiving cavities may also receive the actual bead, any manner of removably attaching the attachment means to the radio lucent material. The term fixedly removably receive is intended to describe that the attachment means may be easily attached for surgery and easily removed once the physician no longer requires the hand to be restrained. The attachment means may also be is made of a radio lucent material. In certain cases it may be desirable to have a hand shaped portion with less than five digits, for example with three or four digits portions. The radio lucent material may be any shape for the part being operated on. For example, it may be desirable to work on the toes of a patient, which would require a similar shape, but each of the digits would be of similar lengths. FIG. 3 depicts an embodiment where one end (11) of the attachment means (12) is fixedly attached to the radio lucent portion (10). In this way, the attachment means cannot be lost or misplaced.

The present invention provides a radio lucent method of stabilizing a portion of a patient during surgery, the method comprising the step of: providing a portion of a patient (e.g. 30) for operating on; placing a radio lucent portion (10) on an operating field; providing at least one attachment means (12) having a flexible elongated portion (10) and at least one bead (16) attached to the flexible elongated portion (10); the radio lucent portion (10) having a multitude of raised portions (22, 24, 26) forming a multitude of receiving cavities (30, 32 and 34) the receiving cavities shaped to removably receive a portion of the attachment means; placing the portion of a patient (for example, the hand (30) on the radio lucent portion (10); removably attaching the portion of the patient to the radio lucent portion by placing at least one attachment means around a portion of a patient and removably attaching at least a portion of the attachment means to one of the multitude of receiving cavities (30, 32 and 34).

According to another embodiment a radio lucent kit for stabilizing a portion of a patient during surgery is provided, the kit comprising: at least one attachment means (12) having a flexible elongated portion (14) and at least one bead (16) attached to the flexible elongated portion (14); a radio lucent portion (10), the radio lucent portion having a multitude of raised portions (e.g. 22, 24, 26 and 28) forming a multitude of receiving cavities (30, 32 and 34), the receiving cavities shaped to removably receive at least a portion of the attachment means (12).

It should be understood that the foregoing relates to preferred embodiments of the invention and that modifications may be made without departing from the spirit and scope of the invention as set forth in the following claims.

We claim:

1. An apparatus for stabilizing a portion of a patient, said apparatus comprising:
at least one attachment means having a flexible elongated portion and at least one bead attached to said flexible elongated portion;
a radio lucent portion, said radio lucent portion having a multitude of raised portions forming a multitude of receiving cavities, each of said receiving cavities shaped to removably receive at least a portion of said attachments means.

2. An apparatus as in claim 1, wherein said attachment means is made of a radio lucent material.

3. An apparatus as in claim 1, wherein said radio lucent portion is hand shaped having at least three digit portions.

4. An apparatus as in claim 1, wherein said receiving cavities are shaped to fixedly removably receive each of said beads to provide stabilization of said hand of said patient.

5. An apparatus as in claim 1, further comprising at least one slit along a side of said radio lucent portion.

6. An apparatus as in claim 1, wherein one end of said attachment means is fixedly attached to said radio lucent portion.

7. A radio lucent method of stabilizing a portion of a patient during surgery, said method comprising the step of:
providing a portion of a patient for operating on;
providing at least one attachment means having a flexible elongated portion and at least one beads attached to said flexible elongated portion;
placing a radio lucent portion on an operating field, said radio lucent portion having a multitude of raised portions forming a multitude of receiving cavities, said receiving cavities shaped to removably receive at least a portion of an attachment means;
placing said portion of a patient on said radio lucent portion;
removably attaching said portion of said patient to said radio lucent portion by placing at least one attachment means around said portion of a patient and removably attaching at least a portion of said attachment means to one of said multitude of receiving cavities.

8. An apparatus as in claim 7, wherein said attachment means is made of a radio lucent material.

9. An apparatus as in claim 7, wherein said radio lucent portion is hand shaped and has at least three digit portions.

10. An apparatus as in claim 7, wherein said receiving cavities are shaped to fixedly removably receive each of said beads to provide stabilization of the hand of said patient.

11. An apparatus as in claim 7, further comprising at least one slit along a side of said radio lucent portion.

12. An apparatus as in claim 7, wherein one end of said attachment means is fixedly attached to said radio lucent portion.

13. A radio lucent kit for stabilizing a portion of a patient during surgery, said kit comprising:
at least one attachment means having a flexible elongated portion and at least one bead attached to said flexible elongated portion;
a radio lucent portion, said radio lucent portion having a multitude of raised portions forming a multitude of receiving cavities, said receiving cavities shaped to removably receive a portion of said attachment means.

14. A radio lucent kit as in claim 13, wherein said attachment means is made of a radio lucent material.

15. A radio lucent kit as in claim 13, wherein said radio lucent portion is hand shaped and has at least three digit portions.

16. A radio lucent kit as in claim 13, wherein said receiving cavities are shaped to fixedly removably receive each of said beads to provide stabilization of said hand of said patient.

17. A radio lucent kit as in claim 13, further comprising at least one slit.

18. A radio lucent kit as in claim 17, wherein said at least one slit is along a side of said radio lucent portion.

19. A radio lucent kit as in claim 13, wherein one end of said attachment means is fixedly attached to said radio lucent portion.
